(19) United States
(12) Patent Application Publication
(10) Pub. No.: US 2007/0141583 A1
(43) Pub. Date: Jun. 21, 2007

(54) Methods of rapid chromatin immunoprecipitation

(76) Inventors: Weiwei Li, New York, NY (US); Jessica M. Li, New York, NY (US)

Correspondence Address: Jessica M. Li, 160 Broadway, #707, New York, NY, 10038, US

(21) Appl. No.: 11/313,014

(22) Filed: Dec. 20, 2005

(51) Int. Cl. C12Q 1/68; C12N 1/08

(52) U.S. Cl. 435/6; 435/270

(57) ABSTRACT

This invention is related to a method for rapidly identifying regions of the genome to which specific proteins bind, or identifying specific proteins bound to a region of the genome in vivo.

### CROSS-REFERENCE TO RELATED APPLICATIONS

Not applicable

### STATEMENT REGARDING FEDERALLY SPONSORED RESEARCH OR DEVELOPMENT

Not applicable

### REFERENCE TO A MICROFICHE APPENDIX

Not applicable

### BACKGROUND OF THE INVENTION

1. Field of the Invention

This invention is related to a method for rapidly identifying regions of the genome to which specific proteins bind, or identifying specific proteins bound to a region of the genome in vivo.

2. Description of the Related Art

Protein-DNA interactions are widely involved in a variety of molecular processes of living cells such as single transduction, gene transcription, chromosome segregation, DNA replication and recombination, and epigenetic silencing of genes (Ptashne M and Cann A. Genes and signals, Cold spring Harbor laboratory Press, 2001, Das, P. M., Ramachandran, K. venWart, J., Signal, R. Biotechniques, 37: 961-969, 2004.). Identifying the genetic targets of DNA binding proteins and knowing the mechanisms of protein-DNA interaction are important for understanding cellular process. The techniques such as electrophoretic mobility shift assay (EMSA) and reporter gene assay have been developed to analyze direct interactions between protein and DNA (Garner, M. M. and Revzin, A. Nucleic Acids Res, 9: 3047-3059, 1981, Revzin, A. BioTechniques, 7: 346-354, 1989, Kelce, W. R., Stone, C. R., Laws, S. C. et al. Nature, 375: 581-585). These technologies have an advantage for analyzing the binding of different transcription factors to specific DNA consensus sequences located in the gene promoters in vitro. However these technologies can not be used for analyzing DNA-protein interactions in a living cell, which is more a complex environment than that in vitro. Although in vivo footprinting may measure protein-DNA interactions in a living cell, the complex creating the footprint is not clearly identified.

Chromatin immunoprecipitaton (CHIP) offers an advantageous tool for studying protein-DNA interactions (Kuo, M. H., Allis, C. D., Methods, 19: 425-433, 1999, Bernstein, B. E., Humphrey, E. L, Liu, C. L., Schreiber, S. L., Methods Enzymol, 376: 349-360, 2004). Unlike EMSA and report gene assays, CHIP allows for detecting that a specific protein binds to the specific sequences of a gene in living cells. For example, measurement of the amount of histone H3 methylation at lysine 9 associated with a specific gene promoter region under various conditions can be achieved through a CHIP-PCR assay. A CHIP method mainly includes the following steps: (1) fixation of cells with formaldehyde to crosslink protein to DNA; (2) harvest of chromatin or protein-DNA complex; (3) precipitation of protein-DNA complex with specific antibodies; and (4) reversal of crosslinked protein-DNA complex and purification of DNA. However the currently used CHIP method has several drawbacks. The most critical weakness of the current CHIP method is time consuming. According to the current CHIP method, it requires 3-4 days to finish its procedure from protein-DNA crosslink to purification of DNA. In addition, the current CHIP method uses suspended beads to capture antibody/complex and centrifugation-dependent wash, which makes the procedure to be labor-intensive and to have low throughput. Thus a more rapid and efficient CHIP method is still needed for overcoming problems of existing methods to improve the analysis of in vivo protein-DNA interactions.

### BRIEF SUMMARY OF THE INVENTION

The present invention provides a CHIP method and kit to rapidly identify regions of the genome to which specific proteins bind, or identifying specific proteins bound to a region of the genome in vivo comprising the steps of:
1) crosslinking protein to DNA in living cells or fresh tissues with formaldehyde.
2) harvesting protein-DNA complex
3) co-precipitating protein-DNA complex with the affinity antibodies.
4) capturing affinity antibodies bound to protein-DNA complex with a second capturing agent coated on a solid support apparatus at an appropriate temperature for the appropriate period.
5) reversing crosslinked complex with a high salt solution containing protein degradation enzymes.
6) purifying DNA with a DNA affinity material.
7) identifying DNA associated with proteins.

Thus the invention allows a rapid and efficient CHIP to be achieved. The invention is based on the finding that protein G or protein A coated on a solid support apparatus can more rapidly and conveniently bind to antibody/protein/DNA complex at room temperature than suspended protein G or protein A beads. The invention is also based on the finding that a high salt solution containing protein degradation enzymes can rapidly reverse crosslinked protein-DNA complex and effectively reduce degradation of DNA at high temperature. Therefore the method presented in this invention significantly overcomes the weaknesses existing in the prior technologies and enables a rapid and efficient CHIP available for identifying regions of the genome to which specific proteins bind, or identifying specific proteins bound to a region of the genome in vivo.

### BRIEF DESCRIPTION OF THE DRAWINGS

FIG. 1 shows a diagram of the rapid CHIP process. The process involves the crosslink of protein to DNA in living cells, harvest of protein-DNA complex and shearing of DNA, the binding of protein-DNA complex to the capturing antibody, capture of antibody/protein/DNA complex with protein G coated on microwells, reversal of crosslinked protein/DNA complex, and DNA purification with DNA affinity materials.

FIG. 2 shows a real time-PCR analysis of rapid CHIP. CHIP was performed using the anti-RNA polymerase II to specifically immunoprecipitate crosslinked DNA-RNA polymerase II. Normal mouse IgG was used as a negative control. The precipitated antibody/DNA/protein complex is captured with protein G coated on microwells. The crosslinked DNA was reversed, purified, and subjected to a real time quantitative PCR using primers specific to the GAPDH promoter. The experiment was carried out as described in Example 1.

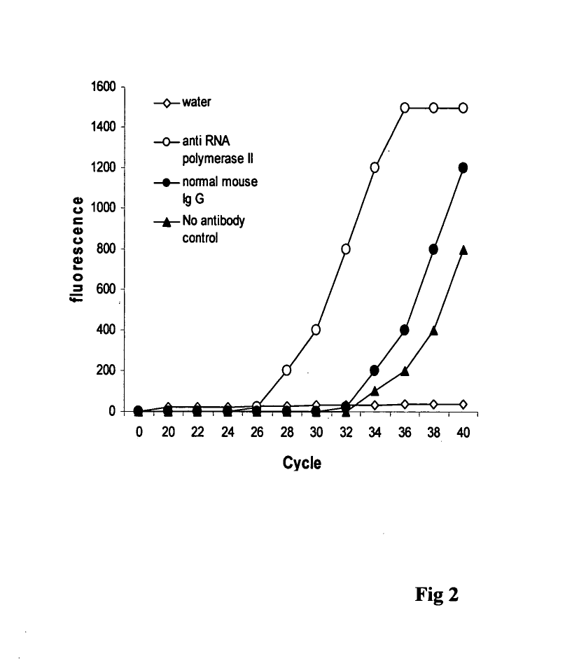

FIG. 3 shows a comparison between rapid CHIP method of this invention and conventional CHIP method. The experiment was carried out as described in Example 2. (—) rapid CHIP; (---) conventional CHIP

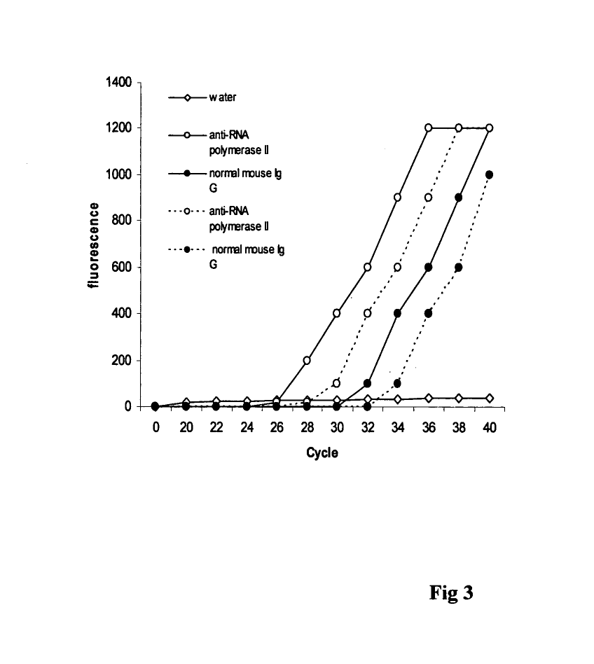

### DETAILED DESCRIPTION OF THE INVENTION

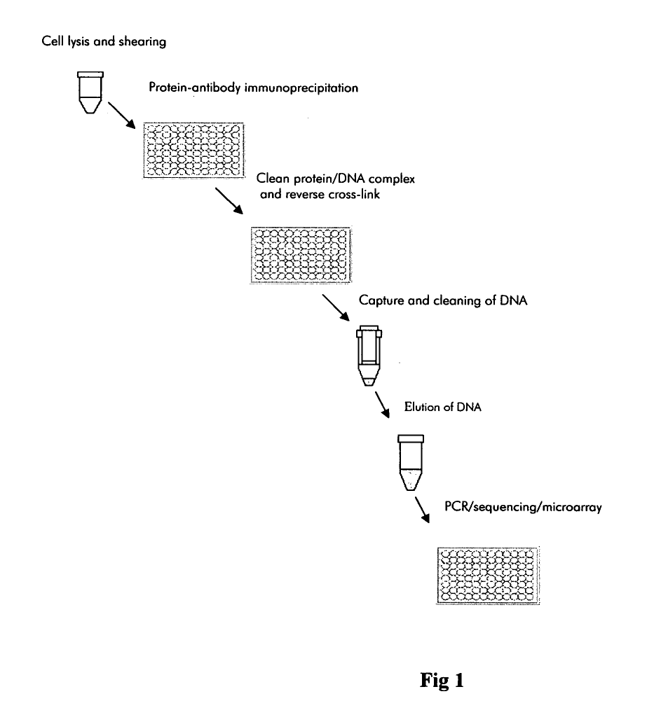

The present invention provides a method and kit to rapidly identify regions of the genome to which specific proteins bind, or identifying specific proteins bound to a region of the genome in vivo. A basic outline of the method presented in this invention is described in FIG. 1. This method is particularly useful for rapidly completing a CHIP assay in a short time. This method is also particularly useful for identifying specific regions to which a target protein binds in a high throughput format.

According to the method of this invention, cells or tissue fragments are first fixed with formaldehyde to crosslink protein-DNA complex. Cells can be incubated with formaldehyde at room temperature or at 37° C. with gentle rocking for 5-20 min, preferably for 10 min. Tissue fragments may need a longer incubation time with formaldehyde, for example 10-30 min, preferably 15 min. The concentration of formaldehyde can be from 0.5 to 10%, preferably 1% (V/V). Once crosslinking reaction is completed, an inhibitor of crosslink agents such as glycine at a molar concentration equal to crosslink agent can be used to stop the crosslinking reaction. An appropriate time for stopping the crosslinking reaction may range from 2-10 min, preferably about 5 min at room temperature. Cells can then be collected and lysed with a lyses buffer containing a sodium salt, EDTA, and detergents such as SDS. Tissue fragments should be homogenized before lysing. For example, disaggregating of tissue fragments can be performed by stroking 10-50 times, depending on tissue type, with a Dounce homogenizer. Cells or the homogenized tissue mixture are then mechanically or enzymatically sheared to yield an appropriate length of the DNA fragment. Usually, 200-1000 bp of sheared chromatin or DNA is required for CHIP assay, especially for that of mapping a specific binding site of a protein. Mechanical shearing of DNA can be performed by nebulization or sonication, preferably sonication. Enzymatical shearing of DNA can be performed by using DNAse I in the presence of Mn salt, or by using micrococcal nuclease in the presence of Mg salt to generate random DNA fragments. The conditions of crosslinked DNA shearing can be optimized based on cells, and sonicator equipment or digestion enzyme concentrations.

Once DNA shearing is completed, cell debris can be removed by centrifugation, and supernatant containing DNA-protein complex is collected and used for immunoprecipitation. At the immunoprecipitation step, cell lysate and an antibody are added to a microwell coated with protein G, or protein A, or protein A/G. Protein G or protein A can be coated on a microwell using the methods as described in prior art or using a carbonate buffer (pH 9.6) with a 1-2 hour or overnight incubation at room temperature. Protein G or protein A can be modified to retain their two IgG-binding domains and to eliminate their albumin and cell surface binding domains so that nonspecific binding can be reduced while the Fc region of IgGs can be efficiently maintained. Protein G would be preferably chosen since its affinity for many more mammalian IgGs is greater than protein A. Cell lysate can be pre-cleared with protein G agarose for 30-60 min and then incubated with antibody in a microwell coated with protein G for 0.5-4 h at room temperature, preferably 1-2 h. Alteratively, a antibody may first bind with protein G coated on a microwell for 30-90 min, preferably 60 min and then cell lysate is added to the microwell. Under this condition, antibody/DNA-protein complex can be efficiently formed and captured by protein G. Compared to the immunoprecipitation step of conventional CHIP assay, in which protein G or protein A conjugates with agarose or sepharose beads, the immunoprecipitation of this invention has several advantages: (1) interaction time of DNA-protein/antibody/capture agents is sharply reduced because of an increased binding rate of antibody to DNA-protein complex at room temperature and no centrifugation required during the washing.; (2) reproducibility and stability are increased since no beads are used and no or little complex will be washed out during the washing; and (3) a high throughput immunoprecipitation can be achieved through a microwell apparatus such as a 96-well plate.

DNA-protein complex captured on a microwell can be washed with a high stringency buffer to eliminate non-covalent interactions. A high stringency buffer may contain 20-50 mM Tris-HCl (pH 8.0), 1-5 mM EDTA, 01-0.5% of SDS, 0.5-1 M NaCl, and 0.5-1% triton x-100. DNA-protein complex captured on a microwell can also be washed with 1×PBS containing 0.5% of tween-20, or 100 mM sodium phosphate containing 200 mM NaCl and detergents such as Tween-20 or Triton X-100.

Crosslinked DNA-protein complex can be reversed after the washing. The buffer for crosslink reversal can be optimized to maximize reversal of the crosslinks and minimize DNA degradation resulting from chemical, biochemical and thermodynamic action. According to the method of this invention, the reversing buffer consists of two parts. The first part of the buffer contains EDTA, SDS, and proteinase K, which should efficiently degrade proteins complexed with DNA and prevent degradation of DNA by nucleases such as DNAse I. The second part contains both sodium and potassium salts with a high concentration. Sodium chloride at 1 M of concentration or potassium chloride at 0.5 M has been demonstrated to efficiently reduce DNA degradation from chemical and thermodynamic action (Marguet, E. Forturre, P, Extremophiles, 2: 115-122, 1998) and increase the reversing rate of formaldehyde crosslinks. The first part of the reversing buffer can be first added to the sample to incubate at 50-75° C., preferably at 65° C., for 5-30 min, preferably for 15 min. The second part of the buffer is then added to continuously incubate at 50-85° C., preferably at 65-75° , for an additional 0.5-4 h, preferably for 0.5-1.5 h. An advantage of this reversing buffer is that the time required for reversal of crosslinks is significantly shortened. Another advantage is that DNA degradation can be maximally prevented.

Once reversal of crosslinked DNA-protein complex is completed, DNA is captured and cleaned. The reversed DNA can be captured by a solid matrix selected from silica salt, silica dioxide, silica polymers, glass fiber, celite diatoms, and nitrocellulose in the presence of high concentrations of non-chaotropic salts. It is preferred, according to this invention, that DNA is captured with an apparatus comprised of a column pre-inserted with a silica gel, a silica membrane, or a silica filter. It is further preferred, according to this invention, that a column is a micro-spin column which fits a 1.5 or 2.0 ml micro-centrifuge tube, and the combination of the column and the micro-centrifuge tube further fits inside a table-top microcentrifuge. A binding buffer consisting of non-chaotropic salts at concentrations from 1 M to 5 M can be added to the DNA sample, and the mixed binding buffer-DNA solution can then be transferred to silica matrix. The DNA-bound silica matrix is washed by adding a washing buffer preferably comprised of a buffered solution containing 50-90% of ethanol. The DNA is then eluted from the column and collected into a capped microcentrifuge tube. An elution solution could be DEPC-treated water or TE buffer (10 mM Tris-HCL, pH 8.0 and 1 mM EDTA).

According to this invention, all of the components for cell lyses, immunoprecipitation, reversal of crosslinks, and DNA purification are commercially available. This invention also provides a kit containing all components required for rapid performance of a CHIP assay. The kit includes: (a) a cell lyses and nucleic acid shearing buffer comprising a Tris-HCl, a detergent, a sodium salt, and proteinase inhibitors; (b) an antibody as the first capturing agent; c). a microwell strip or microwell plate coated with protein G or protein A as the second capturing agent; (d) a nucleic acid-protein complex washing system comprising a low salt solution and a high salt solution; (e) a crosslinked complex reversing solution comprising a SDS, a sodium salt, a potassium salt, a EDTA, and a proteinase K; (f) a nucleic acid isolating system comprising an apparatus with a pre-inserted solid matrix and a isolating buffer containing sodium chloride; g) a DNA elution buffer comprising Tris-HCl, TE, or water, and (h) an instruction for conducting an assay according to the method of this invention. In one embodiment, the kit further comprises selected components to meet the requirements for using cultured monolayer cells, suspension cells, and fresh or frozen tissues including those from clinical samples.

It has been discovered that the use of the method of this invention is able to drastically reduce the time required for immuoprecipitation in assays. It has been also discovered that the use of the method of this invention is able to prevent degradation of DNA in process of crosslinking reversal. It has been further discovered that the use of the method of this invention enables a CHIP assay to be performed in a high throughput format and can be completed within 3-5 h with excellent reproducibility.

The method of this invention for rapid CHIP assay is further illustrated in the following examples:

### EXAMPLE 1

The experiment was carried out to detect whether a specific protein binds to the specific sequences of a gene in living cells.

Antibody binding to the assay plate: Wash plate wells twice with wash buffer (100 mM sodium phosphate, 200 mM NaCl, pH 7.4 and 1% Triton X 100). Dilute various antibodies with wash buffer to 10 μg per ml and add 100 μl of diluted antibody to the each well. Normal mouse Ig G was used as the negative control and anti-RNA polymerase II as the positive control. Cover the plate and incubate at room temperature for 60-90 min. The plate was then washed 3-4 times with wash buffer.

Cell fixation and lysis: Hela cells were grown to 70%-80% confluence on a 100 mm plate, then trypsinized and collected into a 15 ml conical tube. After washing with PBS, cells were suspended in 9 ml of fresh culture medium containing 1% formaldehyde (final concentration) and incubated at room temperature (20-25° C.) for 10 min on a rocking platform (50-100 rpm). 1 ml of glycine (1.25 M) was added and incubated for 5 min to stop formaldehyde crosslink. Cells were washed with PBS and lysed for 10 min in lyses buffer containing 0.5% SDS, 2 mM EDTA, 50 mM Tris-HCl, 150 mM NaCl, 1% Triton X-100, and protease inhibitors. Cells were further lysed by sonication to shear DNA. 5 μl of sonicated cell lysate were removed for agarose gel analysis. 200-1000 bp DNA fragments are usually achieved by sonicating 3-4 pulses of 10-12 seconds each at level 2 using a Branson Microtip probe followed by a 30-40 second rest on ice between each pulse. Cell debris was removed by centrifuging at 12,000 rpm for 10 min at 4° C. and supernatant was collected.

Immunoprecipitation: Cell supernatant was diluted with 1×TE containing 150 mM NaCl and 1% Triton X-100 at a 1:1 ratio. 5 μl of diluted supernatant were removed to a vial as “input” DNA and remaining diluted supernatant were aliquoted and transferred to each well having bound antibodies. The sample was incubated at room temperature (22-25° C.) for 1-2 h on a rocking platform (50-100 rpm). Each well was washed 6 times with wash buffer.

Crosslink reversal: 40 ul of reversing buffer 1 (first part) containing 50 mM Tris-HCl, 0.5% of SDS, 10 mM EDTA, and 0.2 mg/ml of proteinase K were added to each well and incubated with crosslinked DNA-protein complex bound to the well for 15 min at 65° C. 40 μl of reversing buffer 2 (second part) containing 10 mM EDTA, 2.5 M NaCl, and 1 M KCl were then added and incubated for an additional 60-90 minutes at 65° C.

DNA purification: 200 μL of DNA binding buffer containing 5 M NaCl were added to each sample well and the mixed solution containing DNA was transferred to a column apparatus with inserted DNA capture filter. The mixed solution passed through the column in a receiver tube by centrifugation. DNA was eluted from the DNA capture filter.

Real-time PCR analysis: The DNA purified from chromatin immunoprecipitation was analyzed by real-time PCR using primers specific for the GAPDH promoter. PCR was carried out in 25 μl of reaction mixture according to the manufacturer's instructions. PCR reaction mixture was initially incubated at 94° C. for 4 min and then incubated as the following program for 40 cycles: 94° C. for 30 seconds, 55° C. for 30 seconds and 72° C. for 30 seconds. The amplification was observed in the anti-RNA polymerase II CHIP as early as at 27th cycle, while amplification in the normal mouse IgG control and in the no antibody control appeared at the 32^{nd} and the 33^{rd} cycle, respectively. The amplification of control DNA (20 ng/rxn) occurred at the 24^{rd} cycle.

### EXAMPLE 2

The experiment was carried out to compare the effect of the CHIP method of this invention to that of conventional CHIP.

Antibody binding to the assay plate: Wash plate wells twice with wash buffer (100 mM sodium phosphate, 200 mM NaCl, pH 7.4 and 1% Triton X 100). Dilute various antibodies with wash buffer to 10 μg per ml and add 100 μl of diluted antibody to the each well. Normal mouse Ig G was used as the negative control and anti-RNA polymerase II as the positive control. Cover the plate and incubate at room temperature for 60-90 min. The plate was then washed 3-4 times with wash buffer.

Hela cells were grown to 70%-80% confluence on a 100 mm plate, then trypsinized and collected into a 15 ml conical tube. After washing with PBS, cells were suspended in 9 ml of fresh culture medium containing 1% formaldehyde (final concentration) and incubated at room temperature (20-25° C.) for 10 min on a rocking platform (50-100 rpm). 1 ml of glycine (1.25 M) was added and incubated for 5 min to stop formaldehyde crosslink. Cells were washed with PBS and lysed for 10 min in lyses buffer containing 0.5% SDS, 2 mM EDTA, 50 mM Tris-HCl, 150 mM NaCl, 1% Triton X-100, and protease inhibitors. Cells were further lysed by sonication to shear DNA. 5 μl of sonicated cell lysate were removed for agarose gel analysis. 200-1000 bp DNA fragments are usually achieved by sonicating 3-4 pulses at 10-12 seconds each at level 2 using a Branson Microtip probe followed by a 30-40 second rest on ice between each pulse. Cell debris was removed by centrifuging at 12,000 rpm for 10 min at 4° C., and supernatant was collected.

Immunoprecipitation: Cell supernatant was diluted with 1×TE containing 150 mM NaCl and 1% Triton X-100 at a 1:1 ratio. 5 μl of diluted supernatant were removed to a vial as “input” DNA and remaining diluted supernatant were aliquoted and divided into two parts: the first part was transferred to each plate well having bound antibodies and second part was transferred to each vial. For the first part, the sample was incubated at room temperature (22-25° C.) for 1-2 h on a rocking platform (50-100 rpm). Each well was then washed 6 times with wash buffer. For the second part, cell lysate in each vial was pre-cleared by adding salmon sperm DNA/protein G agarose. 1 μg of normal mouse IgG (serving as negative control) and 1 μg of anti-RNA polymerase II antibody were added to pre-cleared lysate in the vials, respectively. One vial having cell lysate but having no antibody was set as the blank control. The mixture was rotated at 4° C. overnight. Once immunoprecipitation was finished, the beads in each vial were washed twice with a buffer containing 0.1% SDS, 20 mM Tris-HCl, 1 mM EDTA, 125 mM NaCl, and 1% Triton X-100, washed twice with a buffer containing 0.1% SDS, 50 mM Tris-HCl, 1 mM EDTA, 1 M NaCl, and 1% Triton X-100; and washed once with 1×TE buffer. Each wash was completed in turn by resuspending, rotating, and centrifuging.

Crosslink reversal: For the method of this invention, 40 μl of reversing buffer 1 (first part) containing 50 mM Tris-HCl, 0.5% of SDS, 10 mM EDTA, and 0.2 mg/ml of proteinase K were added to each well and incubated with crosslinked DNA-protein complex bound to the well at 65° C. for 15 min. 40 μl of reversing buffer 2 (second part) containing 10 mM EDTA, 2.5 M NaCl, and 1 M KCl were then added and incubated at 65° C. for an additional 60-90 minutes. For conventional CHIP, DNA-protein complex were first eluted by incubating with an elution buffer containing 0.5% SDS and 0.1 M NaHCO₃ at room temperature for 15 min. The elution containing DNA-protein complex was collected by centrifugation. To reverse crosslinked DNA, the following steps were carried out: 8 ul of 5 M NaCl was added to 200 ul of elution and the mixture was incubated at 65° C. for 4 h. 4 μl of 0.5 M EDTA, 8 μl of 1 M Tris-HCl and 1 μl of proteinase K (10 mg/ml) were then added to the mixture and incubated at 45° C. for 1 h.

DNA purification: 200 μl of DNA binding buffer containing 5 M NaCl were added to each sample well or vial containing reversed DNA mixture and the mixed solution was transferred to a column apparatus with inserted DNA capture filter. The mixed solution passed through the column in a receiver tube by centrifugation. DNA was eluted from the DNA capture filter.

Real-time PCR analysis: The DNA purified from chromatin inmmunoprecipitation was analyzed by real-time PCR using primers specific for the GAPDH promoter. PCR was carried out in 25 μl of reaction mixture according to the manufacturer's instructions. PCR reaction mixture was initially incubated at 94° C. for 4 min and then incubated as the following program for 40 cycles: 94° C. for 30 seconds, 55° C. for 30 seconds, and 72° C. for 30 seconds. The amplification was observed at the 27th cycle in the anti-RNA polymerase II CHIP using the method of this invention, while the amplification occurred at the 29^{th} cycle in the anti-RNA polymerase II CHIP using the conventional method. The normal mouse IgG control was amplified at the 32^{nd} and the 33^{rd} cycle, respectively, for rapid CHIP using the method of this invention and conventional CHIP. The amplification of control DNA (20 ng/rxn) occurred at 24^{th} cycle.

What is claimed is:

1. A method of identifying interaction of a nucleic acid with a protein in the form of a kit comprising steps of:
a) incubating cells containing a complex comprises said nucleic acid and said protein with a reversible crosslinking agent at an appropriate concentration to crosslink said complex.
b) stopping crosslink of said complex with an inhibitor of said crosslinking agent at an appropriate concentration.
c) lysing said cells and shearing said nucleic acid
d) capturing said first capturing agent with second capturing agent coated on a solid support apparatus at the appropriate temperature for an appropriate period.
e) capturing said crosslinked complex from said lysate with first capturing agent bound to second capturing agent under conditions that said crosslinked complex bind to said capturing agent.
f) reversing said crosslinked complex with a reversing solution at the appropriate temperature for an appropriate period.
g) isolating said nucleic acid from said protein with an isolating buffer and a nucleic acid affinity material.
h) identifying said nucleic acid.

2. The method according to claim 1 wherein said cells are mammalian cells or eukaryotic cells.

3. The method according to claim 1 wherein said cells are primary cell isolates or cultured cells.

4. The method according to claim 1 wherein said crosslinking agent is formaldehyde at an appropriate concentration of from 0.5% to 10%.

5. The method according to claim 1 wherein said an appropriate concentration of formaldehyde incubated with cells is 1%.

6. The method according to claim 1 wherein said an inhibitor is glycine at an appropriate concentration of from 0.1 M to 1 M.

7. The method according to claim 1 wherein said a concentration of glycine is 0.125 M.

8. The method according to claim 1 wherein said shearing of said nucleic acid is mechanical or enzymatic shearing.

9. The method according to claim 1 wherein said first capturing agent is an antibody selectively binding said protein with high affinity.

10. The method according to claim 1 wherein said second capturing agent coated on a solid support apparatus is a binding protein that binds first capturing agent with high affinity.

11. The method according to claim 1 wherein said a solid support apparatus is a microwell, a microwell strip, or a microwell plate.

12. The method according to claim 1 wherein, in step (e), said an appropriate temperature is from 4° C. to 37° C., preferably 15° C. to 25° C.

13. The method according to claim 1 wherein, in step (e), said an appropriate period is from 30 minutes to 4 hours, preferably 1 hour to 2 hours.

14. The method according to claim 1 wherein said a reversing solution consisting of at least an SDS in an amount of from 0.1 to 2%, a sodium salt in an amount of from 0.2 M to 2.5 M, a potassium salt in an amount of from 0.1 to 1 M and a proteinase K at a concentration of from 50 μg/ml to 500 μg/ml.

15. The method according to claim 1 wherein, in step (f), said an appropriate temperature is from 50° C. to 95° C., preferably 65° C. to 75° C.

16. The method according to claim 1 wherein, in step (f), said an appropriate period is from 30 minutes to 4 hours, preferably 1 hour to 2 hours.

17. The method according to claim 1 wherein said an isolating buffer comprises a non-chaotropic salt selected from sodium chloride, lithium chloride, potassium chloride, magnesium chloride, sodium phosphate, lithium phosphate, potassium phosphate, and magnesium phosphate.

18. The method according to claim 1, wherein said a isolating buffer comprises sodium chloride in an amount of from 1 M to 6 M.

19. The method according to claim 1 wherein said a nucleic acid affinity material is a solid matrix selected from the group consisting of celite diatoms, silica polymers, silica dioxide, glass fiber and nitrocellulose.

20. The method according to claim 1 wherein said the kit comprises:
a) a cell lyses and nucleic acid shearing buffer comprising a Tris-HCl, a detergent, a sodium salt and proteinase inhibitors.
b) an antibody as the first capturing agent
c) a protein G or protein A as the second capturing agent which is coated on a solid support apparatus
d) a nucleic acid-protein complex washing system comprising a low salt solution and a high salt solution.
e) a crosslinked complex reversing solution comprising an SDS, a sodium salt, a potassium salt, an EDTA, and a proteinase K.
f) a nucleic acid isolating system comprising an apparatus with a pre-inserted solid matrix and an isolating buffer containing sodium chloride.
g) a DNA elution buffer comprising Tris-HCl, TE or water.
h) an instruction for conducting an assay according to the method of this invention.
